(19) United States
(12) Patent Application Publication
(10) Pub. No.: US 2017/0266102 A1
(43) Pub. Date: Sep. 21, 2017

(54) GEL POLISH COMPOSITION

(71) Applicant: VALIKA COSMETICS LTD., Seongnam-si (KR)

(72) Inventors: Weon Hoe HUH, Seoul (KR); Han Joo KIM, Seongnam-si (KR)

(21) Appl. No.: 15/501,539

(22) PCT Filed: Mar. 14, 2016
(86) PCT No.: PCT/KR2016/002497

§ 371 Date: Feb. 3, 2017

(30) Foreign Application Priority Data
Oct. 15, 2015 (KR) 10-2015-0143791

(51) Int. Cl. A61K 8/898; A45D 29/00; A61K 8/19; A61Q 3/02; A61K 8/04

(52) U.S. Cl. CPC A61K 8/898; A61Q 3/02; A61K 8/042; A61K 8/19; A45D 29/00; A61K 2800/81

(57) ABSTRACT

Provided is a gel polish composition comprising (A) a multifunctional silicone urethane (meth)acrylate oligomer, (B) a reactive monomer, and (C) a photoinitiator. The multifunctional silicone urethane (meth)acrylate oligomer (A) consists of siloxane moieties, urethane moieties, and (meth)acrylate moieties. Also provided is a method of using the gel polish composition.

### TECHNICAL FIELD

The present invention relates to a gel polish composition, and more specifically to a radiation curable gel polish composition having excellent oxygen permeability and gloss.

### BACKGROUND ART

Nail polishes are lacquers that are applied to the fingernails and toenails of humans to decorate and protect the nail plates. Gel polishes are cured under UV radiation and are preferably used to strengthen the solidity and aesthetic appearance of natural nails and minimize the required frequency of nail polishing.

Gel polishes are long-lasting nail polishes. Like general polishes, gel polishes are coated on nails and are not dried until cured under UV light or LED lamps. Since gel polish compositions can last at least 2 weeks after curing, it saves time occurring in case of applying nail polish well frequently.

Since curing is carried out under an UV lamp, the gel polishes are usually composed of an acrylic, methacrylic or epoxy oligomer and a monomer constituting the crosslinking reaction therewith and various additives. Gel polishes of these compositions are made to form a multilayer coating structure when applied to a nail. The multilayer coating structure consists of triple coating such as a base coat, a color coat (or glitter coat), and a top coat. Conventional nail polishes have the disadvantage that oxygen does not freely permeate through multilayer coating films formed on natural nails, and as a result, the contact with the nails is blocked, causing damage to the nails. There is thus a need for a further improved gel polish composition.

### DETAILED DESCRIPTION OF THE INVENTION

### Problems to be Solved by the Invention

One object of the present invention is to provide a gel polish composition having excellent oxygen permeability.

A further object of the present invention is to provide a gel polish composition which can provide a high gloss gel polish to give aesthetic appearance.

Another object of the present invention is to provide a gel polish composition which reduces nail damage and enhances tissue regeneration.

### Means for Solving the Problems

According to one aspect of the present invention, there is provided a gel polish composition comprising (A) a multifunctional silicone urethane (meth)acrylate oligomer consisting of siloxane moieties, urethane moieties, and (meth)acrylate moieties; (B) a reactive monomer; and (C) a photoinitiator.

According to a further aspect of the present invention, there is provided a method of using the gel polish composition, comprising steps of providing a nail substrate; applying the above gel polish composition to the nail substrate to form at least one coat selected from a base coat, a color coat (or glitter coat), and a top coat; and irradiating the entire surface of the coat with radiation to cure the gel polish composition.

### Effects of the Invention

The gel polish composition of the present invention provides a radiation-curable gel that is viscous enough to coat or extend natural and artificial nails. In addition, the gel polish composition of the present invention can provide coatings with high oxygen permeability and gloss when applied to and cured on natural nails and artificial nails. The coatings can prevent damage to the nails due to their high oxygen permeability. Furthermore, the gel polish composition of the present invention can enhance the regeneration of nail tissue compared to existing nail polishes.

### MODE FOR CARRYING OUT THE INVENTION

The present invention will now be described in detail.

The term “gel” means a radiation-curable composition that includes a photoinitiator, and an ethylenically unsaturated monomer and/or oligomer, having a viscosity suitable for coating natural or artificial fingernails and toenails, or forming an artificial fingernails and toenails or nail extensions, or decorating fingernails and toenails.

The term “(meth)acrylic” refers to acrylic and/or methacrylic and the term “(meth)acrylate” refers to acrylate and/or methacrylate.

The gel polish composition of the present invention comprises (A) a multifunctional silicone urethane (meth)acrylate oligomer consisting of siloxane moieties, urethane moieties, and (meth)acrylate moieties, (B) a reactive monomer, and (C) a photoinitiator. In one embodiment, the gel polish composition may further comprise one or more other components selected from the group consisting of (D) a reactive oligomer, (E) a non-reactive monomer, and (F) additives. Preferably, the gel polish composition of the present invention may further comprise (G) a germanium component.

(A) Multifunctional Silicone Urethane (Meth)acrylate Oligomer

The gel polish composition of the present invention comprises (A) a multifunctional silicone urethane (meth)acrylate oligomer consisting of siloxane moieties, urethane moieties, and (meth)acrylate moieties.

The oligomer (A) is a compound including one or more moieties derived from a hydrolysate of a silane compound of the following Formula 1, or a condensate of the hydrolysate, moieties derived from a compound including an NCO group, and moieties derived from a compound having a (meth)acrylate group:

Si(R^{a})ₙR^{b}₄₋ₙ [Formula 1]

wherein R^{a} is a non-hydrolyzable organic group having 1 to 12 carbon atoms, R^{b} is a hydrolyzable group, and n is an integer from 1 to 3.

For example, the non-hydrolyzable organic group represented by R^{a} may be selected from C₁-C₁₂ alkyl groups, C₆-C₁₂ aryl groups, C₇-C₁₂ arylalkyl groups, and C₇-C₁₂ alkylaryl groups, which may be linear, branched or cyclic, and may be present in combination when R^{a} are present in plurality in the same molecule. The hydrolysis resistance required for R^{a} means that R^{a} remains stable under conditions where R^{b} is hydrolyzable.

The hydrolyzable group represented by R^{b} is generally a group that can be hydrolyzed to form a silanol group or a condensate when heated to 25° C. to 100° C. without a catalyst in the presence of excess water. Examples of such hydrolyzable groups include a hydrogen atom, halogen atoms, C₁-C₁₂ alkoxy groups, amino groups, and C₂-C₁₂ acyloxy groups.

In Formula 1, n is an integer from 1 to 3, preferably 1 or 2, and more preferably 2.

Conditions for obtaining a hydrolysate of the silane compound of Formula 1 or a condensate of the hydrolysate are not particularly limited, and for example, the condensate of the desired hydrolysate may be obtained by the following procedure. First, if needed, the silane compound of Formula 1 can be diluted with any suitable solvent, such as ethanol, 2-propanol, acetone or butyl acetate. The solution is added with water and an acid or a base as a catalyst necessary for the reaction of the silane compound. The mixture is stirred to complete the hydrolytic polymerization of the silane compound. For example, the acid may be hydrochloric acid, acetic acid or nitric acid and the base may be ammonia, triethylamine, cyclohexylamine, tetramethylammonium hydroxide or potassium hydroxide (KOH).

Specifically, the compound including an NCO group may be an isocyanate selected from aromatic isocyanates, aliphatic isocyanates having an aromatic ring, aliphatic isocyanates, and mixtures thereof.

Specific examples of isocyanates suitable for use in the present invention include isophorone diisocyanate, hexamethylene diisocyanate, trimethyl hexamethylene diisocyanate, 4,4′-methylene dicyclohexyl diisocyanate, toluene diisocyanate, methylene diphenyl diisocyanate, polymeric methylene diphenyl diisocyanate, tetramethylxylylene diisocyanate, triisocyanurate, isocyanatoethyl methacrylate, isophorone diisocyanate trimer, hexamethylene diisocyanate trimer, hexamethylene diisocyanate biuret, and hexamethylene diisocyanate uretdione. Isocyanate-terminated prepolymers prepared from polyester, polyether or other hydroxyl functional materials may also be used. Mixtures of materials containing isocyanate groups may also be used.

Specifically, the compound having a (meth)acrylate group (acrylic monomer) may be an acrylic monomer having one or more hydroxyl groups, i.e. a hydroxy (meth)acrylate, and specific examples thereof include 2-hydroxyethyl acrylate, 2-hydroxypropyl acrylate, 2-hydroxybutyl acrylate, 2-hydroxyethyl methacrylate, 2-hydroxypropyl methacrylate, 1,3-butanediol acrylate, 1,3-butanediol methacrylate, 1,4-butanediol acrylate, 1,4-butanediol dimethacrylate, ethylene glycol methacrylate, 1,6-hexanediol acrylate, trimethylpropane tri(meth)acrylate, pentaerythritol triacrylate, and glycerol propoxylate triacrylate, which may be used alone or as a mixture of two or more thereof.

A specific example of the compound (A) may be represented by the following Formula 2:

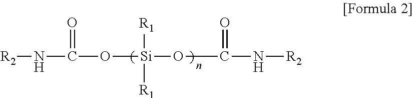

wherein each R₁ is a C₁-C₁₂ alkyl group, each —C(O)NH—R₂ is a group that has a (meth)acrylic group and is derived from an aliphatic or aromatic isocyanate compound, with the proviso that the isocyanate compound has 2 or 3 isocyanate groups, one of which is bonded to the siloxane group, and a (meth)acrylate compound having 1 to 3 (meth)acrylic groups is bonded to each of the other (1 or 2) isocyanate groups non-bonded to the siloxane group, and n is from 10 to 200.

The compound (A) may be prepared by any suitable method in the art. Specifically, the compound (A) is prepared by the following procedure. First, a hydroxyl group end-capped polysiloxane is placed in a flask equipped with a condenser and a stirrer and an isocyanate is added dropwise for 30 minutes. The amount of the isocyanate added is determined considering the hydroxyl equivalent depending on the number of functional groups of the final silicone urethane (meth)acrylate. An organotin or amine catalyst is added in an amount of 0.01 to 1% by weight based on the total weight of the mixture. The resulting mixture is slowly stirred at 50° C. and a (meth)acrylate is added thereto. Stirring is continued for 2 hours, affording the compound (A) having a weight average molecular weight of 5,000 to 20,000. The amount of the (meth)acrylate used is determined considering the isocyanate equivalent depending on the number of functional groups in the final silicone urethane (meth)acrylate.

A more specific example of the compound (A) represented by Formula 2 may be represented by the following Formula 3:

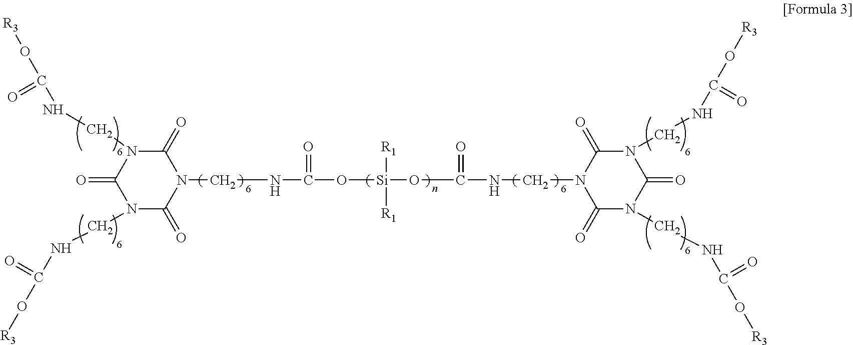

wherein each R₃ is a group derived from a hydroxy (meth)acrylate monomer having 1 to 3 acrylic groups and R₁ and n are as defined in above Formula 2.

The compound of Formula 3 is an oligomer including siloxane, trifunctional isocyanate, and acrylate as constitutional moieties. The compound of Formula 3 is prepared by the following procedure. First, a hydroxyl group end-capped siloxane is allowed to react with a trifunctional isocyanate. At this time, one of the isocyanate groups reacts with one of the hydroxyl groups present in the siloxane. As a result, tetrafunctional silicone isocyanate intermediates are formed in which a total of four isocyanate functional groups remain left and right. At this time, when the monofunctional acrylate monomer is reacted with the remaining four isocyanate functional groups, tetrafunctional silicone urethane acrylate having four acrylate functional groups on the left and right is obtained. Reaction of the remaining four isocyanate functional group with difunctional acrylate monomers will result in an octa-functional silicone urethane acrylate, or reaction of the remaining four isocyanate functional group with trifunctional acrylate monomers will result in a dodeca-functional silicone urethane acrylate.

The compound (A) may be used in an amount of 20 to 90% by weight, preferably 25 to 85% by weight, more preferably 30 to 85% by weight based on the total weight of the composition. If the amount of the compound (A) is less than the lower limit defined above, the resulting coating may tend to be brittle. Meanwhile, if the amount of the compound (A) exceeds the upper limit defined above, poor curing may occur.

The molecular weight of the compound (A) is determined taking into consideration the desired characteristics of the composition in terms of oxygen permeability, gloss, curing properties, and coating film physical properties. The weight average molecular weight of the compound (A) is preferably from 5,000 to 20,000. If the compound (A) has a weight average molecular weight lower than 5,000, the crosslinking density of the compound (A) becomes high at the time of curing, and the resulting coating film may tend to be brittle after curing. Meanwhile, if the weight average molecular weight of the compound (A) exceeds 20,000, the resulting coating film becomes flexible, causing poor scratch resistance to cause poor curing.

The presence of the silicone-based reactive oligomer allows the gel polish composition of the present invention to have a high oxygen permeability and a high gloss, unlike the prior art.

The siloxane bond (Si—O—Si) length in the silicone molecule is longer than the carbon-carbon bond length in a general organic compound. The silicon-oxygen bond angle in the siloxane is greater than the carbon-carbon bond angle in a general organic compound. With these dimensions, the use of the silicone oligomer forms a large network structure in a nail coating, ensuring high oxygen permeability of the coating. In addition, the inherent smoothness of the silicone makes the coating surface smoother, leading to high gloss of the coating.

(B) Reactive Monomer

The gel polish composition of the present invention includes (B) a reactive monomer. The reactive monomer is a compound that can be polymerized by the action of a photoinitiator. The reactive monomer may be a (meth)acrylate-based polymerizable monomer. That is, the reactive monomer may be a monofunctional or multifunctional acrylic acid or methacrylic acid compound having at least one carbon-carbon unsaturated double bond.

Alternatively, the reactive monomer may be a monomer containing at least one free radical polymerizable group in the molecule. Preferably, the reactive monomer is a hydroxyl-containing monomer.

Typical examples of reactive monomers suitable for use in the present invention include acrylic acid and methacrylic acid esters (herein referred to as (meth)acrylates). Specific, non-limiting examples of mono(meth)acryloyl esters include methyl (meth)acrylate, ethyl (meth)acrylate, hydroxypropyl (meth)acrylate, butyl (meth)acrylate, hydroxyethyl (meth)acrylate, butoxyethyl (meth)acrylate, diethylaminoethyl (meth)acrylate, 2-ethylhexyl (meth)acrylate, ethoxyethyl (meth)acrylate, t-butyl aminoethyl (meth)acrylate, methoxyethylene glycol (meth)acrylate, phosphoethyl (meth)acrylate, methoxy propyl (meth)acrylate, methoxy polyethylene glycol(meth)acrylate, phenoxyethylene glycol (meth)acrylate, phenoxypolyethylene glycol (meth)acrylate, 2-hydroxy-3-phenoxypropyl (meth)acrylate, 2-(meth)acryloxyethylsuccinic acid, 2-(meth)acryloylethylphthalic acid, 2-(meth)acryloyloxypropylphthalic acid, stearyl (meth)acrylate, isobornyl (meth)acrylate, 3-chloro-2-hydroxypropyl (meth)acrylate, tetrahydrofufuryl (meth)acrylate, (meth)acrylamide, and allyl monomers. Specific, non-limiting examples of difunctional (meth)acryloyl esters include 1,4-butanediol di(meth)acrylate, 1,6-hexanediol di(meth)acrylate, 1,9-nonanediol di(meth)acrylate, 1,10-decanediol di(meth)acrylate, neopentyl glycol di(meth)acrylate, 2-methyl-1,8-octanediol di(meth)acrylate, glycerin di(meth)acrylate, ethylene glycol di(meth)acrylate, triethylene glycol di(meth)acrylate, polyethylene glycol di(meth)acrylate, propylene glycol di(meth)acrylate, polypropylene glycol di(meth)acrylate, ethoxylated propylene glycol di(meth)acrylate, ethoxylated polypropylene glycol di(meth)acrylate, polyethoxyprophoxy di(meth)acrylate, ethoxylated bisphenol A di(meth)acrylate, propoxylated bisphenol A di(meth)acrylate, propoxylated ethoxylated bisphenol A di(meth)acrylate, bisphenol A glycidyl methacrylate, tricyclodecanedimethanol di(meth)acrylate, glycerin di(meth)acrylate, ethoxylated glycerin di(meth)acrylate, bisacrylamide, bisallyl ether, and allyl (meth)acrylate. Examples of tri- and/or higher (meth)acryloyl esters include trimethylol propane tri(meth)acrylate, ethoxylated glycerin tri(meth)acrylate, ethoxylated trimethylolpropane tri(meth)acrylate, ditrimethylol propane tetra(meth)acrylate, pentaerythritol tri(meth)acrylate, pentaerythritol tetra(meth)acrylate, propoxylated pentaerythritol tetra(meth)acrylate, ethoxylated pentaerythritol tetra(meth)acrylate, dipentaerythritol hexa(meth)acrylate, and ethoxylated isocyanuric acid tri(meth)acrylate.

Examples of preferred hydroxyl-containing monomers include 2-hydroxyethyl acrylate, 2-hydroxyethyl methacrylate, diethylene glycol monoacrylate, diethylene glycol monomethacrylate, glycerol (meth)acrylate, glycerol di(meth)acrylate, sorbitol (meth)acrylate, sorbitol di(meth)acrylate, sorbitol tri(meth)acrylate, 2-hydroxypropyl acrylate, 2-hydroxypropyl methacrylate, 3-hydroxypropyl acrylate, 3-hydroxypropyl methacrylate, tetraethylene glycol mono(meth)acrylate, pentaethylene glycol mono(meth)acrylate, dipropylene glycol monomethacrylate, dipropylene glycol monoacrylate, dipentaerythritol pentaacrylate, dipentaerythritol pentamethacrylate, pentaerythritol triacrylate, pentaerythritol trimethacrylate, caprolactone (meth)acrylate, polycaprolactone (meth)acrylate, polyethylene oxide mono(meth)acrylate, polypropylene oxide (meth)acrylate, ditrimethylol propane tetra(meth)acrylate, carbohydrate based (meth)acrylic monomers, and hydroxyl alkyl (meth)acrylamides, for example, n-methylol acrylamide. Most preferred hydroxyl-containing monomers are hydroxyethyl methacrylate (HEMA) and hydroxypropyl methacrylate (HPMA). Mixtures of two or more hydroxyl-containing monomers may be used.

A compound having at least one free radical polymerizable group includes not only a single component but also a mixture of polymerizable monomers. Thus, combinations of two or more materials containing free radical polymerizable groups may be used.

In addition of these, other reactive monomers include, but are not limited to: multifunctional urethane acrylate compounds obtained by reacting an alicyclic compound having a straight-chain alkylene group and two or more isocyanate groups with a compound having one or more hydroxyl groups and 3, 4 or 5 acryloyloxy and/or methacryloyloxy groups in the molecule; and epoxy acrylates.

The above-mentioned reactive monomers may be used alone or in combination thereof.

The compound (B) is used in an amount of 5 to 70% by weight, preferably 5 to 65% by weight, more preferably 5 to 50% by weight, based on the total weight of the composition. If the amount of the compound (B) is less than the lower limit defined above, poor curing may occur. Meanwhile, if the amount of the compound (B) exceeds the upper limit defined above, the resulting coating may tend to be brittle or the composition may become thin and flow down during coating.

(C) Photoinitiator

The composition of the present invention contains a photoinitiator. The photoinitiator plays a role in initiating the polymerization reaction of monomers that can be cured by visible light, ultraviolet rays or deep-ultraviolet radiation. The photoinitiator may be a radical initiator or a cationic initiator and its type is not particularly limited. The photoinitiator may be selected from the group consisting of acetophenone-based, benzophenone-based, benzoin-based, benzoyl-based, xanthone-based, triazine-based, halomethyloxadiazole-based, rofin dimer-based photopolymerization initiators, and mixtures thereof.

Examples of photoinitiators suitable for use in the present invention include benzyl ketones, monomeric hydroxyl ketones, polymeric hydroxyl ketones, alpha-amino ketones, acyl phosphine oxides, phosphinates, metallocenes, benzophenones, and benzophenone derivatives. Specific examples include, but are not limited to, 1-hydroxy-cyclohexylphenyl ketone, benzophenone, 2-benzyl-2-(dimethylamino)-1-(4-(4-morpholinyl)phenyl)-1-butanone, 2-methyl-1-(4-methylthio)phenyl-2-(4-morpholinyl)-1-propanone, diphenyl-(2,4,6-trimethylbenzoyl)phosphine oxide, phenyl bis(2,4,6-trimethylbenzoyl)phosphine oxide, benzyl dimethyl ketal, isopropylthioxanthone, ethyl(2,4,6-trimethylbenzoyl)phenyl phosphinate, phenyl(2,4,6-trimethylbenzoyl)phenyl phosphinate, 2,2′-azobis(2,4-dimethylvaleronitrile), 2,2′-azobis(4-methoxy-2,4-dimethylvaleronitrile), benzoyl peroxide, lauryl peroxide, t-butyl peroxypivalate, 1,1-bis(t-butylperoxy)cyclohexane, p-dimethylaminoacetophenone, 2-benzyl-2-(dimethylamino)-1-[4-(4-morpholinyl)phenyl]-1-butanone, 2-hydroxy-2-methyl-1-phenyl-propan-1-one, benzyl dimethyl ketal, benzophenone, benzoin propyl ether, diethyl thioxanthone, 2,4-bis(trichloromethyl)-6-p-methoxyphenyl-s-triazine, 2-trichloromethyl-5-styryl-1,3,4-oxodiazole, 9-phenylacridine, 3-methyl-5-amino-((s-triazin-2-yl)amino)-3-phenylcoumarin, 2-(o-chlorophenyl)-4,5-diphenylimidazolyl dimer, 1-phenyl-1,2-propanedione-2-(o-ethoxycarbonyl)oxime, 1-[4-(phenylthio)phenyl]-octane-1,2-dione-2-(O-benzoyloxime), o-benzoyl-4′-(benzmercapto)benzoyl-hexyl-ketoxime, 2,4,6-trimethylphenylcarbonyl-diphenylphosphonyloxide, hexafluorophosphoro-trialkylphenylsulfonium salt, 2-mercaptobenzimidazole, and 2,2′-benzothiazolyl disulfide. These photoinitiators may be used alone or as a mixture thereof.

The compound (C) is used in an amount of 0.1 to 12% by weight, preferably 1 to 12% by weight, more preferably 3 to 10% by weight, based on the total weight of the composition. If the amount of the compound (C) is less than the lower limit defined above, curing may be delayed, leaving uncured portions. Meanwhile, if the amount of the compound (C) exceeds the upper limit defined above, a temperature rise may occur during curing, making a user feel hot or causing yellowing of the coating.

(D) Reactive Oligomer

The composition of the present invention may optionally further include a reactive oligomer that is copolymerizable with the silicone-based reactive oligomer (A). The reactive oligomer may be selected from the group consisting of urethane (meth)acrylates, which are different from the compound (A), polyester (meth)acrylate oligomers, polyether (meth)acrylate oligomers, epoxy (meth)acrylate oligomers, modified oligomers thereof, and mixtures thereof. The modification may be accomplished by fatty acid oils, amines, thiols, and fluorine. The kind of the reactive oligomer may be suitably selected depending on the desired physical properties of the composition.

The reactive oligomer may be an oligomer of a (meth)acrylate-based polymerizable material or a monomer containing at least one free radical polymerizable group in the molecule. The (meth)acrylate-based polymerizable material is the same as explained in the compound (B)

The reactive oligomer is a reactive unsaturated compound and may be an oligomer of at least one monomer selected from the group consisting of, but not limited to, ethylene glycol di(meth)acrylate, propylene glycol di(meth)acrylate, diethylene glycol di(meth)acrylate, triethylene glycol di(meth)acrylate, 1,6-hexanediol di(meth)acrylate, polyethylene glycol di(meth)acrylate, polypropylene glycol di(meth)acrylate, glycerin tri(meth)acrylate, trimethylolpropane tri(meth)acrylate, pentaerythritol tri(meth)acrylate, a monoesterification product of pentaerythritol tri(meth)acrylate with succinic acid, pentaerythritol tetra(meth)acrylate, dipentaerythritol penta(meth)acrylate, dipentaerythritol hexa(meth)acrylate, a monoesterification product of dipentaerythritol penta(meth)acrylate with succinic acid, caprolactone modified dipentaerythritol hexa(meth)acrylate, pentaerythritol triacrylate hexamethylene diisocyanate (a reaction product of pentaerythritol triacrylate and hexamethylene diisocyanate), tripentaerythritol hepta(meth)acrylate, tripentaerythritol octa(meth)acrylate, bisphenol A epoxy acrylate, and ethylene glycol monomethyl ether acrylate.

The molecular weight of the reactive oligomer is determined taking into consideration the characteristics (e.g., adhesiveness and flexibility) of the resulting coating layer. Preferably, the reactive oligomer has a weight average molecular weight of 1,000 to 10,000. If the weight average molecular weight of the reactive oligomer is lower than 1,000, the adhesiveness or flexibility of the coating layer may deteriorate. Meanwhile, if the weight average molecular weight of the reactive oligomer is higher than 10,000, poor curing may occur.

The compound (D) is used in an amount of 0.1 to 50% by weight, preferably 1 to 30% by weight, more preferably 5 to 15% by weight, based on the total weight of the composition. The amount of the compound (D) can be suitably selected taking into consideration the adhesiveness and flexibility of the resulting coating layer. If the amount of the compound (D) is less than the lower limit defined above, the effect of adding the compound (D) is difficult to expect. Meanwhile, if the amount of the compound (D) exceeds the upper limit defined above, poor curing may occur.

(E) Non-Reactive Monomer

A general radiation curable fingernail/toenail coating includes only solid components without liquid components. Preferably, the gel polish composition of the present invention may be prepared by mixing the components (A), (B), (C), and optionally (D) with a non-reactive monomer. The non-reactive monomer is compatible and unreactive with the above-described components. Any non-reactive monomer known in the field of gel polish compositions may be used in the present invention. The non-reactive monomer is preferably volatile.

A suitable non-reactive monomer may be rapidly volatilized during UV curing, leaving areas with increased porosity in the resulting coating layer. A remover solution can easily enter the coating layer through the porous areas.

Examples of such non-reactive monomers include, but are not limited to, ketones, alkyl acetates, alcohols, alkanes, and alkenes, which may be used alone or as a mixture thereof. The non-reactive monomer may be suitably selected from the group consisting of acetone, ethyl acetate, butyl acetate, isopropyl alcohol, ethanol, butanol, diacetone alcohol, methyl ethyl ketone, hexane, propylene glycol, butyl carbitol, and mixtures thereof.

The content of the non-reactive monomer in the gel polish composition of the present invention is not particularly limited and may be determined, for example, in terms of the coatability and dispersibility of the composition. For example, the non-reactive monomer may be included in such an amount that the solid content is from 50 to 95% by weight, based on the total weight of the composition. As used herein, the term “solid content” refers to the amount of the solid components in the composition from which the non-reactive monomer is excluded.

(F) Additives

The gel polish composition of the present invention may further include one or more additives selected from the group consisting of UV absorbers, polymerization accelerators, UV stabilizers, defoaming agents, leveling agents, thixotropic agents, glitters, and pigments. The roles of these additives are known in U.S. Pat. No. 6,818,207 and WO2013/192515. Each of the additives may be included in an amount of 0.001 to 10% by weight, based on the total weight of the composition.

(G) Germanium Component

In a preferred embodiment, the gel polish composition of the present invention may further include a germanium component.

Germanium (Ge) is a porous mineral belonging to the group of rare earth metals and has 32 electrons. When a heavy metal, a toxic substance or pollutant as foreign matter comes into contact with the electrons of germanium, one of the four outer electrons leaves and binds to the foreign matter. This explains the ability of germanium to decompose or neutralize foreign matter. Germanium has deodorizing effects and antibacterial activity. Another function of germanium is to emit far-infrared radiation that is beneficial to the human body. Once absorbed, far-infrared radiation activates cellular activity to improve metabolic function and promote blood circulation. Due to its antibacterial activity, far-infrared radiation suppresses the propagation of microbes, such as fungi, and is thus beneficial for nail beauty.

Germanium emits anions when the ambient temperature rises to 32° C. or above. Due to this anionic emission, germanium promotes blood circulation upon contact with the human body. Accordingly, germanium is effective in loosening knotted muscles, recovering from fatigue, alleviating pain, inhibiting fatigue substances from accumulating, and enhancing the natural healing power of human beings. Germanium has many functions, including air purification, sterilization, and deodorization, which are associated with the effects of anions. For example, when a slight amount of a germanium component is added to a toenail polish, it can be expected to remove a bad smell from toenails.

The germanium component may include at least one component selected from the group consisting of organogermanium compounds, inorganic germanium compounds, and germanium metal powders.

As the organogermanium compounds, there may be exemplified germanium (IV) tetraethoxide [Ge(C₂H₅O)₄], germanium (IV) tetra-n-butoxide [Ge(C₄H₉O)₄], germanium (IV) tetraisopropoxide [Ge(C₃H₇O)₄], β-carboxyethylgermanium (IV) oxide [(GeCH₂CH₂COOH)₂O₃], tetraethylgermanium (IV) [Ge(C₂H₅)₄], tetrabutylgermanium (IV) [Ge(C₄H₉)₄], and tributylgermanium (IV) hydride [Ge(C₄H₉)₃H]. β-carboxyethylgermanium (IV) oxide [(GeCH₂CH₂COOH)₂O₃] is preferred because it is easily available.

As the inorganic germanium compounds, there may be exemplified germanium monoxide (GeO) and germanium dioxide (GeO₂). Inorganic germanium is a kind of mineral that is partially contained in natural minerals and fossils. A slight amount of inorganic germanium is also found in general soil.

The germanium component is added in an amount of 0.001 to 0.1% by weight, preferably 0.005 to 0.05% by weight, more preferably 0.005 to 0.04% by weight, based on the total weight of the nail polish. If the amount of the germanium component added is less than the lower limit defined above, substantial effects of germanium are difficult to expect. Meanwhile, if the amount of the germanium component added exceeds the upper limit defined above, the germanium component is difficult to dissolve in the composition and phase separation occurs easily, resulting in a deterioration in the stability of the final product over time.

As described above, the compounds (A), (B), and (C) are essential components and the compounds (D), (E), (F), and (G) are optional components for the gel polish composition of the present invention.

The gel polish composition has a viscosity of 1,000 to 50,000 cP, preferably 2,000 to 20,000 cP. If the viscosity of the gel polish composition is less than the lower limit defined above, the gel polish composition may be high fluidity, making it difficult to apply to nails. Meanwhile, if the viscosity of the gel polish composition exceeds the upper limit defined above, the gel polish composition may not be sufficiently spreadable when applied to nails. The term “spreadability” as used herein means that the gel polish composition is smoothly coated on nails.

The composition can be coated on human fingernails and toenails. The composition may also be coated on artificial fingernails and toenails. The radiation curing may be performed using any suitable radiation curing apparatus known in the art of fingernail/toenail coating. The radiation curing conditions are also known in the art. The radiation curing apparatus may be, for example, a UV lamp or LED lamp. The radiation curing of the gel polish composition gives a coating layer having a thickness of 1 μm to 0.5 mm.

The gel polish composition of the present invention can be applied to the formation of at least one coat selected from a base coat, a color coat (or glitter coat), and a top coat.

According to one embodiment of the present invention, a method of using the gel polish composition is provided. The method may include providing a nail substrate, applying the gel polish composition to the nail substrate to form at least one coat selected from a base coat, a color coat (or glitter coat), and a top coat, and irradiating the entire surface of the coat with radiation to cure the coat.

The nail substrate may be a natural or artificial fingernail or toenail.

The base coat is a transparent or white polish agent and is used to help nails strengthen and/or assist in attaching the color coat (or glitter coat) and other decorative materials to nails.

The color coat (or glitter coat) imparts an aesthetic effect to nails and may vary in color and glitter.

The top coat is a transparent polish agent that is used after the color coat is coated on a nail. The top coat protects the color coat (or glitter coat) and forms a solidified barrier to prevent a nail from being broken, scratched or peeled.

The gel polish composition can be cured by UV light to form a cured coat having an oxygen permeability of 250 cm³/m²·day and a gloss of at least 150. The oxygen permeability and the gloss may reach, for example, 910 cm³/m²·day and 172, respectively, but are not limited thereto.

The gel polish composition exhibits a high oxygen permeability and a high gloss when applied to fingernails and toenails. The addition of germanium mitigates damage to nails, ensures a healthy look, reinforces the nail skin, removes an unpleasant smell, and imparts luster to nails due to the ability of germanium to emit far-infrared rays and anions.

The present invention will be embodied by way of the following examples. However, these examples are set forth for illustrative purposes and the present invention is not limited thereto.

### EXAMPLES

### Examples 1-3 and Comparative Examples 1-3

Gel polish compositions were prepared according to the composition shown in Table 1. The silicone urethane (meth)acrylate oligomer was prepared by reacting an isocyanate with the both terminal hydroxysilane in the presence of a catalyst to synthesize a silicone isocyanate intermediate and reacting an acrylate with the silicone isocyanate intermediate. The silicone urethane (meth)acrylate was octafunctional having a weight average molecular weight of 12,000. A commercially available difunctional urethane acrylate oligomer was used as the reactive oligomer. A commercially available monofunctional acrylate was used as the reactive monomer. TPO available from BASF was used as the photoinitiator.

Each of the gel polish compositions was stirred, degassed, coated with a bar coater, and cured under a 24-watt LED lamp for 0.5-1 min to form a coating layer. The physical properties of the coating layer were measured. The results are shown in Table 1.

The oxygen permeability of the coating layer was measured using an Illinois 8003 oxygen permeability tester in accordance with the ASTM F 1927:2014 test method. The gloss of the coating layer was measured using a Horiba gloss meter. The gel polish composition was coated to a thickness of 120 μm on a transparent PET film and cured with UV. The coating was measured for gloss. The gloss of the gel polish was compared with the gloss (180) of the transparent PET film. The viscosity of the gel polish was measured using a Brookfield LV viscometer with spindle No. 64 at 25° C.

The numbers given in Table 1 represent parts by weight of the corresponding components.

TABLE 1

| | | | | Comparative | Comparative | Comparative |
|---|---|---|---|---|---|---|
| Component | Example 1 | Example 2 | Example 3 | Example 1 | Example 2 | Example 3 |
| Silicone urethane | 30 | 60 | 85 | | | |
| (meth)acrylate oligomer | | | | | | |
| Reactive oligomer | 15 | 5 | 5 | 30 | 60 | 85 |
| Reactive monomer | 50 | 30 | 5 | 65 | 35 | 10 |
| Photoinitiator | 5 | 5 | 5 | 5 | 5 | 5 |
| Oxygen permeability | 250 | 400 | 910 | 80 | 150 | 220 |
| (cm³/m² · day) | | | | | | |
| Gloss | 150 | 160 | 172 | 130 | 142 | 145 |
| Viscosity (cps) | 2000 | 7000 | 9000 | 1500 | 3000 | 7000 |

As can be seen from the results in Table 1, the gel polish compositions of Examples 1-3 had the same viscosity range as the gel polish compositions of Comparative Examples 1-3. The oxygen permeability and gloss of the gel polish compositions of Examples 1-3 were higher than those of the gel polish compositions of Comparative Examples 1-3.

### Examples 4-7 and Comparative Examples 4-5

Gel polish compositions were prepared as shown in Table 2. Each of the gel polish compositions was stirred, degassed, coated, and cured under a LED lamp for 1 min. The resulting coating layer was measured for far-infrared emissivity and anion emission. The results are shown in Table 2. The numbers given in Table 2 represent parts by weight of the corresponding components.

TABLE 2

| | | | | | Comparative | Comparative |
|---|---|---|---|---|---|---|
| Component | Example 1 | Example 2 | Example 3 | Example 4 | Example 4 | Example 5 |
| Silicone urethane | 85 | 85 | 85 | 85 | | |
| (meth)acrylate oligomer | | | | | | |
| Reactive oligomer | 5 | 5 | 5 | 5 | 85 | 90 |
| Reactive monomer | 5 | 5 | 5 | 5 | 15 | 5 |
| Photoinitiator | 5 | 5 | 5 | 5 | 5 | 5 |
| Germanium | 0.001 | 0.01 | 0.02 | 0.05 | 0 | 0 |
| Far-infrared emissivity | 0.884 | 0.886 | 0.887 | 0.889 | 0.837 | 0.841 |
| (5-20 μm) | | | | | | |
| Anion emission (count/cc) | 121 | 123 | 126 | 128 | 101 | 104 |
| Appearance | Transparent | Transparent | Transparent | Phase | Transparent | Transparent |
| | | | | separation | | |

(1) Far-Infrared Emissivity Measurement

Far-infrared emissivity was measured using FT-IR according to the KFIA-FI-1005 test method for far-infrared measurement (Korea Far Infrared Association).

(2) Anion Emission Measurement

Anion emission was measured using an apparatus for measuring the number of charged particles according to the KFIA-FI-1042 test method for anion emission measurement (Korea Far Infrared Association).

As can be seen from the results in Table 2, the gel polish compositions of Examples 4-7 emitted higher far-infrared ray emissivity and anion emission compared to the gel polish compositions of Comparative Examples 4-5.

1. A gel polish composition, comprising: (A) a multifunctional silicone urethane (meth)acrylate oligomer consisting of siloxane moieties, urethane moieties, and (meth)acrylate moieties, (B) a reactive monomer, and (C) a photoinitiator.

2. The gel polish composition according to claim 1, wherein the multifunctional silicone urethane (meth)acrylate oligomer is a compound comprising one or more moieties derived from a hydrolysate of a silane compound of the following Formula 1, or a condensate of the hydrolysate, moieties derived from a compound comprising an NCO group, and moieties derived from a compound having a (meth)acrylate group:
Si(R^{a})ₙR^{b}₄₋ₙ [Formula 1]
wherein R^{a} is a non-hydrolyzable organic group having 1 to 12 carbon atoms, R^{b} is a hydrolyzable group, and n is an integer from 1 to 3.

3. The gel polish composition according to claim 1, wherein the multifunctional silicone urethane (meth)acrylate oligomer has a weight average molecular weight of 5,000 to 20,000.

4. The gel polish composition according to claim 1, wherein the reactive monomer is a monomer containing at least one free radical polymerizable group in the molecule.

5. The gel polish composition according to claim 1, wherein the reactive monomer is a (meth)acrylate-based polymerizable monomer.

6. The gel polish composition according to claim 1, wherein the multifunctional silicone urethane (meth)acrylate oligomer (A) is a compound represented by the following Formula 2: 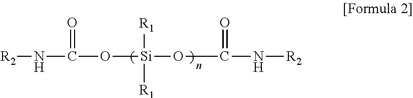
wherein each R₁ is a C₁-C₁₂ alkyl group, each —C(O)NH—R₂ is a group that has a (meth)acrylic group and is derived from an aliphatic or aromatic isocyanate compound, with the proviso that the isocyanate compound has 2 or 3 isocyanate groups, one of which is bonded to the siloxane group, and a (meth)acrylate compound having 1 to 3 (meth)acrylic groups is bonded to each of the other (1 or 2) isocyanate groups non-bonded to the siloxane group, and n is from 10 to 200.

7. The gel polish composition according to claim 1, wherein the compound (A), the compound (B), and the compound (C) are present in amounts of 20 to 90% by weight, 5 to 70% by weight, and 0.1 to 12% by weight, respectively, based on the total weight of the composition.

8. The gel polish composition according to claim 1, further comprising one or more compounds selected from the group consisting of (D) a reactive oligomer, (E) a non-reactive monomer, and (F) additives.

9. The gel polish composition according to claim 8, wherein the reactive oligomer is selected from the group consisting of urethane (meth)acrylates, polyester (meth)acrylate oligomers, polyether (meth)acrylate oligomers, epoxy (meth)acrylate oligomers, modified oligomers thereof, and mixtures thereof.

10. The gel polish composition according to claim 8, wherein the non-reactive monomer is selected from the group consisting of acetone, ethyl acetate, butyl acetate, isopropyl alcohol, ethanol, butanol, diacetone alcohol, methyl ethyl ketone, hexane, propylene glycol, butyl carbitol, and mixtures thereof.

11. The gel polish composition according to claim 8, wherein the additives are selected from the group consisting of UV absorbers, polymerization accelerators, UV stabilizers, defoaming agents, leveling agents, thixotropic agents, glitters, pigments, and mixtures.

12. The gel polish composition according to claim 1, further comprising (G) a germanium component.

13. A method of using the gel polish composition according to claim 1, the method comprising steps of: providing a nail substrate; applying the gel polish composition to the nail substrate to form at least one coat selected from a base coat, a color coat or glitter coat, and a top coat; and irradiating the entire surface of the coat with radiation to cure the coat.

14. The method according to claim 13, wherein the result cured coat has more than 250 cm³/m²·day oxygen permeability and a gloss of at least 150.
